(19) United States
(12) Patent Application Publication
(10) Pub. No.: US 2011/0250688 A1
(43) Pub. Date: Oct. 13, 2011

(54) Three Dimensional Tissue Generation

(75) Inventor: Syed K. Hasan, Lowell, MA (US)

(73) Assignee: Immunotrex Corporation, Lowell, MA (US)

(21) Appl. No.: 13/130,740

(22) PCT Filed: Nov. 24, 2009
(86) PCT No.: PCT/US2009/065718

§ 371 Date: Jun. 27, 2011

Related U.S. Application Data
(60) Provisional application No. 61/117,461, filed on Nov. 24, 2008.

(51) Int. Cl. C12N 5/071

(52) U.S. Cl. 435/395

(57) ABSTRACT

Methods and materials for generating a three-dimensional tissue construct are provided. The method comprises (a) providing a tissue scaffold; (b) providing isolated smooth muscle cells; (c) suspending the isolated cells in hydrogel; (d) ejecting the cell/hydrogel suspension from a printer onto the tissue scaffold; and (e) incubating the cells on the scaffold in media at 37° C.

### TECHNICAL FIELD

The present invention provides compositions and methods for use in generating three dimensional tissue constructs.

### BACKGROUND

The following description provides a summary of information relevant to the present application and is not an admission that any of the information provided or publications referenced herein is prior art to the present application.

Organ transplantation is emerging as a transforming technology. Nonetheless, this technology is limited by the relative scarcity of donors. As of September 2008, nearly 100,000 Americans were awaiting kidney, heart, liver, lung or pancreatic islet cell transplantation in the U.S. alone (http://www.optn.org/data/). Tissue engineering is one potential approach to generate a renewable source of tissues for transplantation. The main theme of tissue engineering is to reproduce the body's architectural and geometric complexity. Using such approaches, tissues might be cultivated, expanded in vitro and re-introduced into the body to replace a damaged part of an organ.

Bladder disease/dysfunction encompasses a wide range in clinical medicine, from typical urinary tract infections to bladder cancer. The total number of new bladder disease/dysfunction cases per year is reported to be 13 million in the USA. Four-hundred million people suffer from bladder diseases world-wide and require partial or total organ reconstruction. Over the past 20 years, medications, behavioral modifications, and surgical interventions have helped in alleviating symptoms associated with bladder dysfunction, but the quality of life for patients still remains poor. Due to the limited success with therapeutic treatments, removal and reconstruction of the entire bladder wall is considered the most effective approach to treat bladder cancer and to reduce mortality. While the gold standard for bladder cancer treatment is to use gastrointestinal segments to reconstruct the bladder wall, this approach involves a range of complications such as metabolic disturbances, stone formation, increased mucus production, and malignant disease.

Bioprinting is an emerging technology to spatially control placement of cells mixed with matrix materials to build 3D tissue structures. The urinary bladder in vivo consists of smooth muscle cells (SMCs) that form SMC-collagen layers. SMC layers are arranged in orthogonal directions to allow optimal contractions during micturition.

Traditional 3D scaffold approaches are not suitable for generating complex tissue structures due to lack of spatial and temporal control of cell seeding. In particular, cells in 2D culture as well as within traditional 3D scaffolds simply do not organize as they do in normal tissue. Specific limitations of most in vitro cultures are that they are 1) not 3D, and 2) with uncontrolled cell seeding. To overcome these limitations micro patterning approaches have been used to induce orientation of cardiac cells on patterned surfaces. However, despite their utility in generating oriented tissues in vitro, the incorporation of such features in 3D scaffolds has not been demonstrated. Scaffolding techniques have traditionally been used to form degradable porous, polymer scaffolds that are subsequently flooded with hydrogel encapsulated cells. Yet seeding cells one after another in a controlled, repeatable fashion is difficult. To address this issue, alternative approaches to build 3D tissues have been proposed such as the use of cell-laden hydrogels, stepwise brick-by-brick building block techniques, and stencils for controlled geometry. Unfortunately, these methods do not offer the ability for high-throughput and repeatable formation of 3D tissue structures and for controlling spatial cell locations within a single block.

The present invention is directed toward overcoming one or more of the problems discussed above, especially in light of the difficulties in uniformly seeding cells within a scaffold.

### SUMMARY OF THE EMBODIMENTS

The present invention provides compositions and methods for use in generating three dimensional tissue constructs. Such constructs can be implanted into a patient.

In some embodiments, methods are provided for generating a three-dimensional tissue construct where the method comprises (a) providing a tissue scaffold; (b) providing isolated smooth muscle cells; (c) suspending the isolated cells in hydrogel; (d) ejecting the cell/hydrogel suspension from a printer onto the tissue scaffold; and (e) incubating the cells on the scaffold in media at 37° C.

These and various other features and advantages of the invention will be apparent from a reading of the following detailed description and a review of the appended claims.

### BRIEF DESCRIPTION OF THE DRAWINGS

FIG. 1 demonstrates a three-dimensional tissue printing and characterization of collagen encapsulated SMCs droplets. (a), Schematic of solenoid valve showing cells and collagen mixture flowing into the valve by constant air pressure. Droplets are generated by way of a pulse generator linked to the solenoid valve. Pulse width (open period of the valve) and frequency (on/off time of the valve) are controlled to generate required volume and to place droplets onto substrate. (b), Image of collagen droplets printed in a line formation on a slide glass. (c), Two layers are shown with a droplet in each layer. (d), Graph, showing number of cells per droplet, cell number increases as initial cell loading concentration was increased (1×10⁶/ml, 5×10⁶/ml, 10×10⁶/ml). (e), Second graph shows variation of droplet placement within a printed line.

FIG. 2(a) illustrates printed droplets in line pattern; FIG. 2(b) illustrates droplets in line pattern within 2 separate layers. Figure (c) shows images of printed lines. Figures (d) and (e) show two printed lines in a cross pattern printed within separate layers. The top and bottom layers are shown focused respectively.

FIG. 3 provides 3D SMC patch schematic and focal images of actual 3D tissue construct. (a) Image of top layer of collagen, (b) top layer of SMCs, (c) separating collagen layer, (d) bottom layer of SMCs, (e) bottom collagen layer. (f) Cell distribution of 2D patch for 1 million, and 10 million cells/ml concentration. Each patch size is 3 mm by 10 mm, (b) μ±σ (average±STD) of each patches are 85±13, 186±77, respectively.

FIG. 4(a) provides quantification of cell distribution within 2D SMC printed patch Day(s) 0,1,2, and 4. Each patch size is 3 mm by 10 mm and the μ±σ (average±STD) for each patch are 85±13, 114±42, and 186±77 respectively. SMC's encapsulated in collagen droplets printed in a unidirectional pattern patch; ˜13000 cells/per line. FIG. 4(b) shows day 2 culture of SMC patch, stained with DAPI (blue) and actin (green) under a light microscope (10×). FIG. 4(c) shows day 7 SMCs stained with DAPI and actin. FIG. 4(d) shows SMCs stained with DAPI (blue) at day 14 in culture. FIG. 4(e) provides SMCs stained with DAPI, connexin-43 (red). The DAPI and connexin-43 staining show cell position and viability.

FIG. 5 is an image of cell printing setup enclosed in a sterile field. The tissue printer setup is run by the Stage Controller, which is given commands by the Computer. The Ejection Setup is controlled by the cell printer, which receives signals from the pulse generator. Period: 80 ms, Pulse Width: 60 μs, all cells encapsulated in 2% collagen, (mean1:11, mean2:28, mean3:54) Illustration of solenoid valve showing the ejection of cell droplets onto a substrate.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art(s) to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

As used herein, the term “hydrogel” is used to mean polymeric matrices that can absorb a substantial amount of water to form gels, wherein “matrices” are three-dimensional networks of macromolecules held together by covalent or noncovalent interactions. Hydrogel materials include but are not limited to collagen and extracellular matrix.

As used herein, the term “printer” is used to mean an apparatus which spatially and temporally controls placement of cells, optionally, mixed with matrix materials.

As used herein, the term “scaffold” is used to mean one or more layers having properties that can be varied to alter the tissue scaffold's features, for example, strength, volume, porosity and durability, as well as, performance. Scaffold materials include biological or synthetic materials. Scaffold materials include but are not limited to collagen, fibrin, chitosan, hyaluronic acid, carbon nanotube, polyester, polylactic acid, polyglycolic acid, and polycaprolactone.

As used herein, the term “seeded” is used to mean initially placed at a predetermined position.

As used herein, the term “smooth muscle cells” is used to mean cells which form or have the potential to form involuntary non-striated muscle.

As used herein, the term “substrate” is used to mean any surface either biological or non-biological onto which cells are or can be seeded.

As used herein, the term “tissue” is used to mean any assemblage of multiple cells.

Described herein are new possibilities for treatment and even possible cures of bladder disease and associated dysfunctions using bioengineering. Recent clinical success in bladder tissue engineering demonstrated the feasibility of combining synthetic polymer scaffolds with host cells harvested from bladder tissue of patients in need of bladder reconstruction. It is considered a very promising technique, since it eliminates some of the complications associated with the conventional bowel segment grafting. However, there are a number of limitations with conventional tissue engineering techniques. For example, it is difficult to seed cells uniformly within a scaffold, and to spatially control tissue micro-architecture, matrix, and cellular placement. These difficulties limit the function of engineered bladder tissues with respect to the mechanical and biological properties of the tissue.

Embodiments in accordance with the present invention include methods and compositions useful in generating 2D and 3D tissue constructs. Embodiments are predicated on the unexpected finding that the use of patternable cell-laden hydrogel droplets permits uniform cell seeding.

Cell printing methods (e.g., laser printing and inkjet printing) permit controlled cell position within 3D constructs. Described herein is a technique of tissue engineering using cell-laden hydrogel droplets with engineered microvasculature features in the bioengineered scaffolds. Use of cell-laden hydrogel when combined with cell printing methods diminishes or eliminates the difficulties associated with other micro fabricated tissue engineering scaffolds such as uniform cell-seeding.

In some aspects, methods are provided for generating a three-dimensional tissue construct. The method comprises providing a tissue scaffold, providing isolated cells, suspending the isolated cells in hydrogel, ejecting the cell/hydrogel suspension from a printer onto the tissue scaffold, and incubating the cells on the scaffold at 37° C. The ejecting step can be performed one or more times to produce the desired cell thickness. In some embodiments, a cell-free hydrogel is placed on the scaffold prior to ejecting the cell/hydrogel suspensions onto the scaffold.

In one embodiment, the tissue printer setup is run by a stage controller which determines the horizontal XY positioning of the stage upon which the substrate, optionally including one or more scaffolds, is supported. The stage controller receives positioning commands from a computer. Drops are generated by way of a pulse generator linked to a solenoid valve. The flow of cell/hydrogel suspension though the solenoid valve is mediated by air pressure. Pulse width (open period of the valve) and frequency (on/off time of the valve) are controlled by the pulse generator which controls the required volume and placement of droplets of cell/hydrogel suspension on the substrate.

Cells can be seeded at a concentration sufficient to permit normal growth through cell to cell contacts and production of growth factors and cytokines. For example, cells can be seeded at a concentration of at least about 2.5×10⁵, at least about 5×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, or at least about 1.5×10⁷ cells/mL, or at any concentration in between the enumerated concentrations.

In one embodiment, the cells are suspended in a hydrogel. Any useful hydrogel is contemplated herein, including, for example, collagen and extracellular matrix.

The concentration of hydrogel can be at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, or at least about 100 mg/mL, or any concentration falling between any of the enumerated concentrations.

In one embodiment, the placement of droplets is in a line pattern. The lines of droplets may be organized in parallel or may intersect. The lines may form a cross pattern wherein lines of droplets intersect perpendicularly.

In another embodiment, fractal solutions are used to determine the placement of droplets in order to generate 3D bioengineered tissues and organs. A fractal is a rough or fragmented geometric shape that can be split into parts, each of which is (at least approximately) a reduced-size copy of the whole a property called self-similarity. Roots of mathematical interest in fractals can be traced back to the late 19th Century; however, the term “fractal” was coined by Benoit Mandelbrot in 1975 and was derived from the Latin fractuss meaning “broken” or “fractured.” A mathematical fractal is based on an equation that undergoes iteration, a form of feedback based on recursion. A fractal often has the following features: (1) it has a fine structure at arbitrarily small scales; (2) it is too irregular to be easily described in traditional Euclidean geometric language; (3) it is self-similar (at least approximately or stochastically); (4) it has a Hausdorff dimension which is greater than its topological dimension (although this requirement is not met by space-filling curves such as the Hilbert curve); and (5) it has a simple and recursive definition. Because they appear similar at all levels of magnification, fractals are often considered to be infinitely complex (in informal terms). Natural objects that are approximated by fractals to a degree include clouds, mountain ranges, lightning bolts, coastlines, snowflakes, various vegetables (cauliflower and broccoli), and animal coloration patterns. However, not all self-similar objects are fractals—for example, the real line (a straight Euclidean line) is formally self-similar but fails to have other fractal characteristics; for instance, it is regular enough to be described in Euclidean terms.

The tissue construct can be provided for implantation into a mammal, for example, a human, dog, cat, horse, cow, etc. The tissue construct can be bladder, uterus, reproductive tract, pancreas, liver, gastrointestinal tract, respiratory tract, blood vessel, or other tissue, particularly including smooth muscle tissue.

It has been shown that autologous bladder cells harvested from the host can be cultured on synthetic and/or biological scaffolds and implanted back into animals and humans. In one case, expanded urothelial and bladder SMCs harvested from patients were cultured for up to 8 weeks, then seeded on bladder-shaped scaffolds made of either collagen alone or collagen/poly(glycolic acid) composite. The seeded scaffolds were then implanted back into patients.

The 3D architecture of engineered bladders needs to be improved as the architecture has been shown to govern bladder function. For example, biomechanical and histological analyses performed by the inventors have demonstrated that anisotropic mechanical behavior (approximately 20% stiffer in the longitudinal direction than in the circumferential) found in the normal bladder is strictly correlated to the orientation of smooth muscle layers more biased in the longitudinal direction compared to circumferential (26±6% vs. 14±5%)^{30,31}. Together with the fact that pathological bladder tissues exhibit altered morphology and mechanical isotropy, it can be speculated that the preferred orientation of smooth muscle layers is for optimal function of the bladder. To engineer bladders that are functionally closer to the native bladder, it is necessary to formulate alternative approaches for precise control of 3D cell organization. Provided herein is a bladder tissue using a bottom-up, microscale tissue engineering approach based on cell-laden hydrogel printing. In some aspects, a 3D cell microenvironment is provided which allows for complex cell-cell and cell-matrix interactions. 3D cell patterning apparatus allows efficient cell-matrix deposition with high spatial resolution and uniform initial cell seeding density, while maintaining cell viability and functionality. This is a unique and repeatable tissue printing system to print tissues from collagen encapsulated smooth muscle cells. This technology permits several hundred micrometer scale level control of the smooth muscle 3D cell position and orientation. Rat bladder tissue, for example, can now be generated by this tissue printing technology. The printed bladder can be then be placed into rat models and used to elucidate biochemical signaling pathways needed for cellular architecture.

In some embodiments, the scaffold is made of biological components. Illustratively, the scaffold can be made of collagen, fibrin, chitosan, or hyaluronic acid. In other embodiments, the scaffold is made of synthetic components such as carbon nanotube or linear aliphatic polyester. In other embodiments, the scaffold is made of polylactic acid, polyglycolic acid, or polycaprolactone.

In combination with MRI, PET, and CT scans, three-dimensional scaffolds can be created with any appropriate material.

Various embodiments of the disclosure could also include permutations of the various elements recited in the claims as if each dependent claim was multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

### EXAMPLES

The following examples are provided for illustrative purposes only and are not intended to limit the scope of the invention.

### Example 1

### Methods

### Reconstituted Collagen

Collagen Reconstitution was performed by mixing 250 μl Type I Bovine collagen (MP Biomedicals) with 50 μl Sterile H2O, 50 μl 10× PBS (Gibco) 50 μl FBS (Gibco), 50 μl 1× SMC medium (1640 RPMI, Gibco) and 50 μl 0.1M NaOH (Sigma). Type I collagen was reconstituted by previously mentioned method. Reconstituted collagen was mixed by gentle pipetting without formation of bubbles. Reconstituted collagen was kept at 4° C. before being mixed with rat smooth muscle cells after cells were trypsinized.

### Smooth Muscle Cell Medium Preparation

445 ml of 1× 1640 RPMI (Gibco) plus 50 ml FBS (Gibco) and 5 ml Pen/Strep (Gibco) was sterile filtered (500 mL Express Plus 0.22 μm membrane) once all components were mixed together.

### Tissue Printer

A valve based droplet generator (Hewlett Packard) is an electromechanical device used to control the flow of fluids. Current flows through a metal coil located within the solenoid to generate a magnetic field. This causes displacement of the actuator within the solenoid (Tech Elan). The actuator is attached mechanically to a valve within the solenoid section. The valve then changes states (open/close), to either allow or block the flow of fluids through the solenoid. In most cases a spring is used to return the metal actuator back to its original state once the current is removed from the solenoid valve. A pulse generator was programmed to apply electrical current to solenoid valves at desired rates so that the solenoid valves were uniformly and consistently actuated. An important aspect of the valves is the fluid passing through the valve was kept under constant pressure to ensure that the fluid passed through the valve uniformly when it was actuated.

### Trypsinization of Smooth Muscle Cell Culture

SMC cultures were grown until the flask was 80% confluent (i.e. rat bladder primary smooth muscle cells). After the culture reached confluency the medium was aspirated. The flask was briefly washed with 5 mL PBS (Gibco, 1×) and aspirated off before 1 mL trypsin (Gibco, 1×) was placed on to SMC culture flask and incubated at 37° C. for 1 minute. The flask was then taken out of the incubator and checked using light microscopy for detached smooth muscle cells. Once a majority of cells (>75%) have detached from bottom of flask, a 10 mL pipette and panning technique was used to remove the remaining cells from the flask bottom. 5 mL of pre-warmed media was then added to the trypsinized cells to neutralize the trypsin. Cells were then placed in to a 15m1 conical tube and spun down at 1200 rpm for 1 minute. The resultant supernatant was aspirated off and cells were resuspended in 1 mL fresh pre-warmed SMC medium. Cell counts were performed using trypan blue (Gibco, 1:5 dilution) and a hemacytometer. SMCs were placed into reconstituted collagen mixture, and gently mixed by pipetting up and down; 1 mL of cell/collagen mix was placed into the ejector setup syringe. Multiple samples were printed onto a cell culture dish (60 mm×15 mm, Falcon) for each type of staining. A minimum of 4 samples for each type of stain was done for proper analysis.

### Printing Process

Using the valve based droplet ejector and setup described previously, cells in media were ejected from the ejector. Using a predetermined concentration of cells, either (1 million cells/mL, 5 million cells/mL, or 10 million cells/mL), the 10 mL syringe attached to the ejector was filled with the desired amount cell/collagen suspension. The ejector and collagen were constantly cooled, as the collagen solution can solidify at room temperature. Liquid nitrogen (LN₂) vapor was used to cool the setup, making sure not to freeze the collagen or solenoid valve.

The printing process was adjusted to meet the specifications of various experiments. The ability to eject media of varied cell densities using the droplet ejector was accomplished by adjusting one or more parameters. By increasing the pulse width parameter, the solenoid valve remains open during each actuation cycle longer thus enabling media with greater density to flow through more readily. Droplet size was adjusted in two ways. The pulse width was manipulated so that the valve only remained open for a short period of time resulting in smaller droplets ejected during each actuation. The number of cells per droplet was adjusted by changing the droplet size or the initial loading cell concentration to the ejector, which is proportional to number of encapsulated cells.

### Printing Cell Density Assay

SMC patches (5 mm×5 mm) were printed with two different initial concentrations (1×10⁶/mL and 10×10⁶/mL). Printed patches were imaged daily at 10× and total cells were counted. Cell counts were then placed into excel spreadsheet program for graphical analysis. Counts for Day 0, 1 and 2 were taken and graphed to show cell distribution and cell proliferation.

Staining smooth muscle cells post printing

Printed SMC encapsulated collagen droplets were first allowed to gel at 37° C. for 20 minutes before SMC medium was added to the dishes. Printed SMC's patches were incubated overnight. Cells were allowed to expand and spread; elongated cells were seen under light microscope at (10×). After 24 hrs, medium was aspirated off and printed cells were washed with room temperature PBS (1×). Primary antibody (1:50 dilution, in 1× PBS) was placed on printed cells and incubated at 37° C. for 40 minutes. (Primary antibodies used, actin, connexin 43, mouse monoclonal IgG, Santa Cruz Biotechnology), and DAPI, (Invitrogen). After 40 minutes, primary antibody was washed off with PBS (1×) 3 times, and secondary antibody, (goat anti-mouse IgG FITC, IgG R, 1:50 dilution in PBS 1×) were placed onto printed cells and incubated for 40 minutes. After secondary antibody incubation, excess antibody is washed off with PBS (1×) and then 3 mL PBS (1×) added onto printed lines of SMC's. Printed lines imaged under florescent microscope.

### Live/Dead Stain

Live/Dead Stain (Invitrogen) 0.5 μl of calcian plus 2 μl of ethidium bromodimer into 1 mL of 1× PBS was made and mixed well, by inverting the tube for one minute. After which 1 mL of the live/dead solution was placed onto printed lines. The printed lines were incubated for 10 minutes at 37° C. After incubation printed lines were imaged by using a florescent microscope to check viability of cells.

### DAPI Stain

Printed SMC encapsulated collagen patches were first allowed to gel at 37° C. for 20 minutes before 500 μl of SMC medium was placed onto printed patches. Printed SMCs placed into the incubator overnight. Cells expanded and elongated were seen under the light microscope at (10×). After 24 hrs, medium was suctioned off and printed cells were washed with room temperature PBS (1×) 3 times. DAPI stain (Invitrogen, 5 μg/mL) was placed onto printed SMC's and Incubated for 40 minutes at 37° C., then rinsed 3 times with 1× PBS. After the third rinse, 3 mL of 1× PBS was placed onto cells and the cells were imaged with UV light using a florescent microscope.

### Results

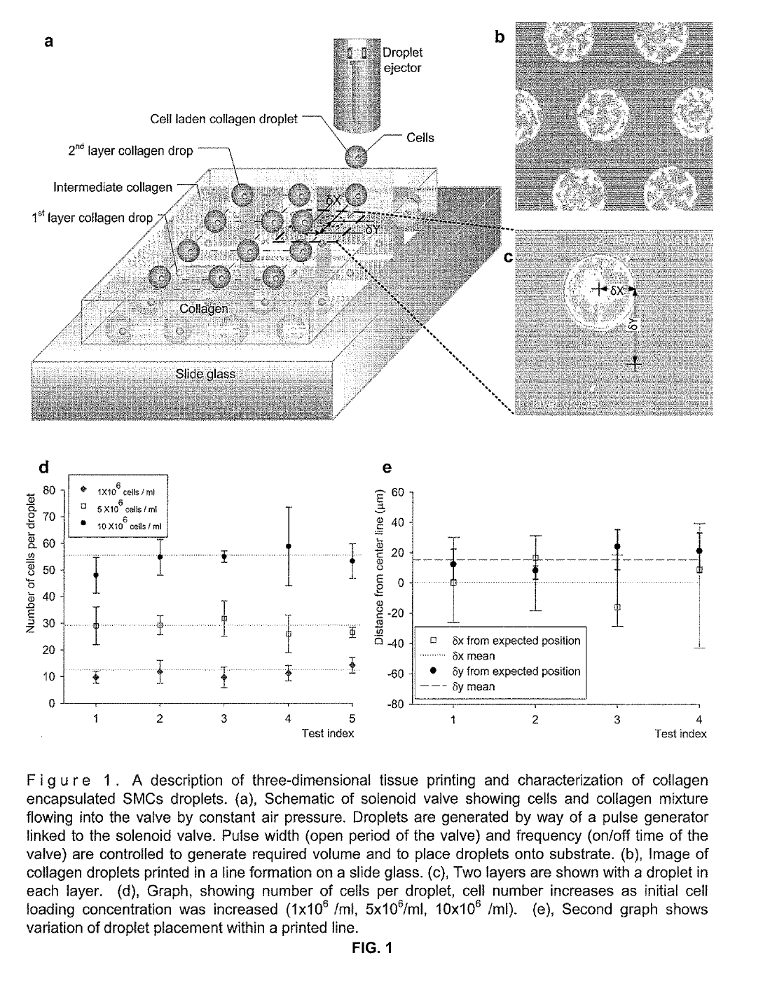

These results demonstrate the feasibility of patterning cell-laden collagen droplets in 3D to assemble tissues in a high-throughput and scalable manner. To spatially and temporally control placement of collagen encapsulated cell droplets, a cell-laden droplet generator was merged with xyz microstage controller to build 3D tissue structures (FIG. 1a). To achieve the temporal and spatial control, the droplet generation and stage movement was unified under single controller software. The system controlled the droplet ejection direction frequency by operating the droplet generator using an electrical on/off signal. The movement of the microstage was synchronized with this droplet generation resulting in 3D patterning capability. This allows the system to deposit a cell-laden hydrogel droplet wherever desired in 3D.

To have a statistically tight control on the cell seeding density, the number of cells per each cell-laden droplet (FIG. 1b) at three cell loading densities was counted. The number cells per droplet increased as the cell loading density to the ejector reservoir was increased. The number of cells that packed in a single droplet did not increase linearly with the loading density, since the droplet size was fixed. It was more difficult to pack more cells into a fixed volume. The mean and standard deviation for number of cells per droplet was investigated to drive an understanding of seeding density. Smaller standard deviations resulted in more uniform seeding density as cells were patterned to create the 3D scaffolds.

Next, to investigate the ability to achieve uniform cell seeding, droplet landing locations on a surface during droplet generation were characterized. The landing locations of the droplets determine the overlap between the deposited droplets, which affects the cell seeding density. To characterize the printing precision, lines of single cell-laden droplets were deposited and the variation in location compared to where the droplets were expected to have landed (FIG. 1c). Droplet ejection directionality is the major determinant of this variation, since no observations were made of vibrations within the system or on the stages to achieve a 20 um x and 30 um y variation. The variation is comparable to a single cell diameter. The system precision suffices to establish a controlled cell seeding density with printing (FIG. 1d).

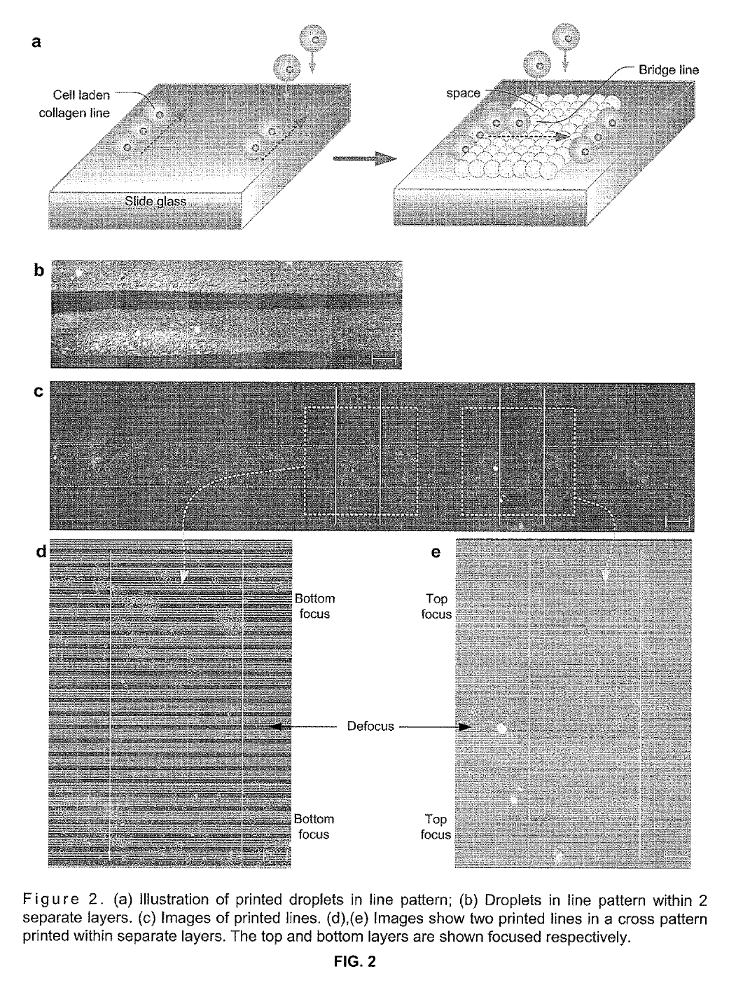

A 10 mm×10 mm cell-laden collagen patch (FIG. 2a) was printed to test the system operation. The patch was created by printing cell-laden collagen droplets onto a Petri-dish substrate. The droplets were observed to land onto the surface and spread to a wider area (<300 um), where the cells were uniformly distributed within the landed droplet. Collagen droplets were used as collagen forms a temperature crosslinkable gel. The substrate temperature was higher than the droplet generator reservoir, enabling the collagen to be in liquid state during droplet generation and gel after it reached the deposition surface. The adjacent droplets gel together and form a single seamless layer due to the temperature crosslinking. The control software set the overlap between the adjacent droplets to 50% (FIG. 2b). This overlapping and gelling droplets enabled a wide area patch.

The printed patches have high viability and functionality. The printed cells were in spherical shape after they were removed from the flask and right after they were mixed with collagen as well as during the printing process. The printing of a 5 mm×5 mm patch consumes 20 seconds indicating the high throughput aspect of the system and explaining why the cells were still in spherical geometry. The cells were observed to adhere and spread within the printed cell-laden collagen layer, indicating cell functionality and viability (FIG. 2c). It was also demonstrated that the cells were viable through live/dead stains and long-term culture. In day 2 and day 7, the printed cells were observed to express actin indicating the cellular functionality after the printing and culturing processes (FIGS. 2c-d). The printed patches were further cultured for 14 days to show the organization and expansion of cells within the printed patches. The 14^{th} day patch expressed connexin-43, which indicated establishment of cell-to-cell communication channels within the printed patch (FIGS. 2e-f).

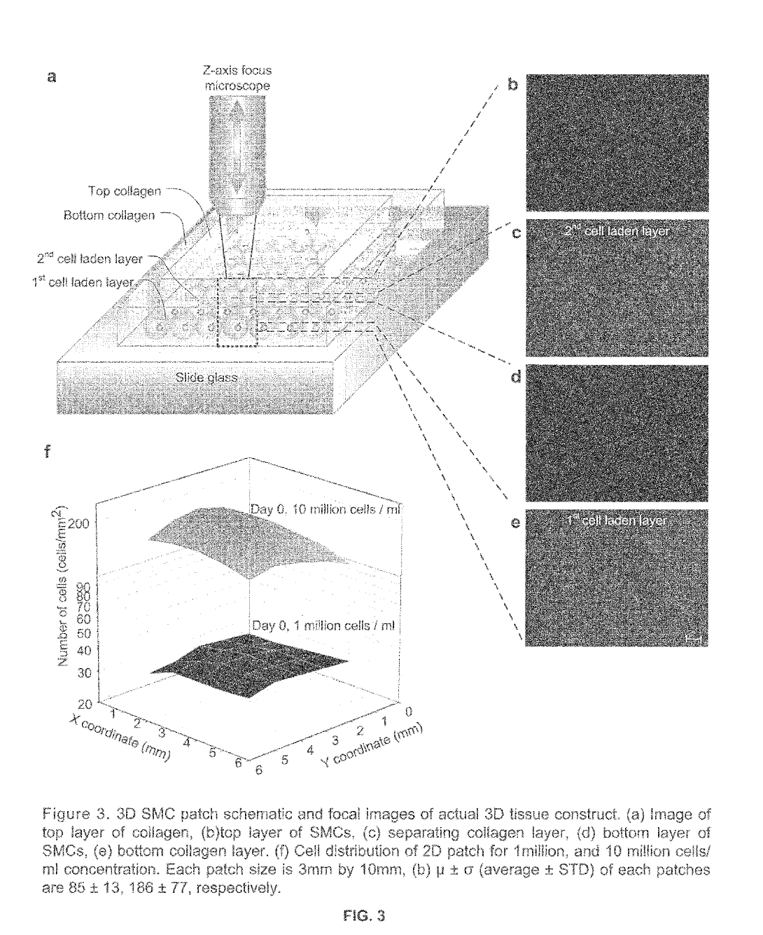

Uniform cell seeding density is critical for tissue engineering, since it controls the average cell-to-cell distances as well as cell-to-cell communication. The overall mechanical behavior of a tissue construct depends on this uniformity. This problem is addressed by the cell-laden droplet printing approach. As shown above, the cell-laden hydrogel droplets have uniform number of cells per droplet at various cell seeding densities. Further, precise control now exists on where these droplets are placed, which look identical and merge conformably together. These capabilities are granted by the printing technology. Cell-laden collagen patches were printed to observe the cell seeding densities within the same layer at three different cell densities. The patches were imaged after the printing and the number of cells in each image throughout the patches was determined. The distribution, uniformity, and variation of cell seeding density by the printing method are shown (FIG. 3a). Higher cell loading density resulted into higher number cells per droplet, which led to a higher number of cells printed per area per patch. Therefore, 10 million cells/ml patch layer has a higher average cell seeding density then the other two cell printing densities (FIG. 3a). Color coding of the top view of these patches reveal the uniformity of cell seeding with the printing technology. The same colors within the printed patch indicate the seeding uniformity, whereas the increased color intensity between the patches indicates the increased cell seeding density, which correlates with the increased number of cells per droplet (FIG. 3b). The side view of the patch is characterized to show the variation in cell seeding density (FIG. 3c). This characterization is important for the tissue printing technology, since it builds the ties between a generated cell-laden droplet and a created tissue construct. Controlling technical parameters such as the cell number per droplet and precise positing of these droplets has a direct impact on the cell seeding density of the printed scaffold, average cell-to-cell distances, which eventually determines the cell-to-cell communication, and functionality of the created tissue construct. This can further lead to enhanced quality of life of the patients, as more functional tissues can be created by this unique technology.

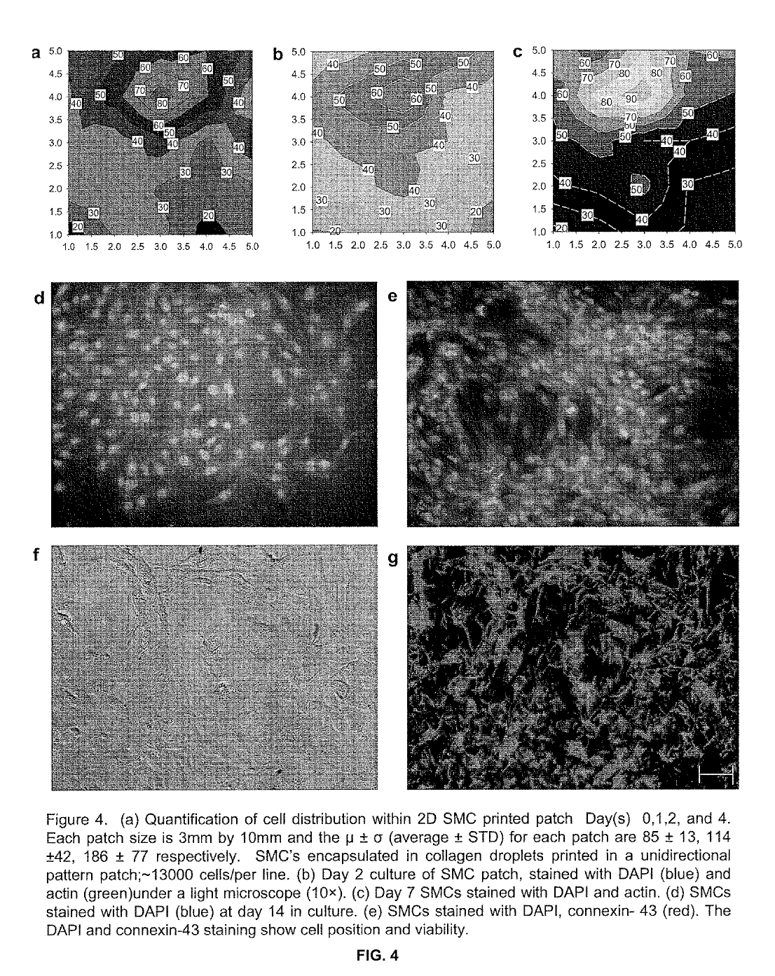

Finally, native bladder tissue comprises a multiple layer smooth muscle cell structure. To generate a printed bladder smooth muscle tissue construct, the cell-laden collagen droplets were patterned on top of the earlier printed layers. This was achieved by first gelling the initial printed layers and then depositing addition cell-laden hydrogel droplets on top of the previously printed layer. For example, a bottom cell-less collagen layer was printed first, and on top of this layer a cell-laden collagen layer was printed. The process was performed three times creating a total of 5 collagen layers (382.5 um thick). To monitor the multiple layers, a motorized system was created that can step the microscope focus and take images that are at its focus (FIG. 4a). The printed 3D multilayer smooth muscle cell-laden collagen tissue construct was stained with DAPI. The focal images show layers with stained cells and without cells. The cell-laden layers (FIGS. 4c-e) show clearly stained circular cellular nuclei, whereas the cell-less collagen layers only show background staining with DAPI.

In conclusion, described herein is an approach that utilizes a 3D patterning technology to assemble micro-scale cell-laden hydrogel droplets creating 3D tissue constructs. This enables high precision, high throughput creation of bioengineered 3D tissues with the proper cell and ECM localization in 3D to mimic the native tissues. This bottom-up printing technology enables a powerful, highly scalable method to create tissue constructs from microscale units of cell-laden hydrogel droplets. The utilization of 3D tissue printing approach confers uniform cell seeding densities in 3D scaffolds, and allows patterning cells in 3D to enable tissue formation. This method permits micro patterning of 3D cell-laden ECM and using well-established hydrogels for long-term culture. This method allows printing of multiple cell types in complex tissue micro architecture including vasculatures printed simultaneously with the 3D tissues. Printing technology provides spatial and temporal control of printing viable and functional cells. It is a novel method used to generate a 3D bladder tissue construct. This technology enables micron-level control of the SMC orientation, and cellular location. The printed 3D bladder tissue constructs are 3 cell layers thick, allowing diffusion of oxygen and nutrients.

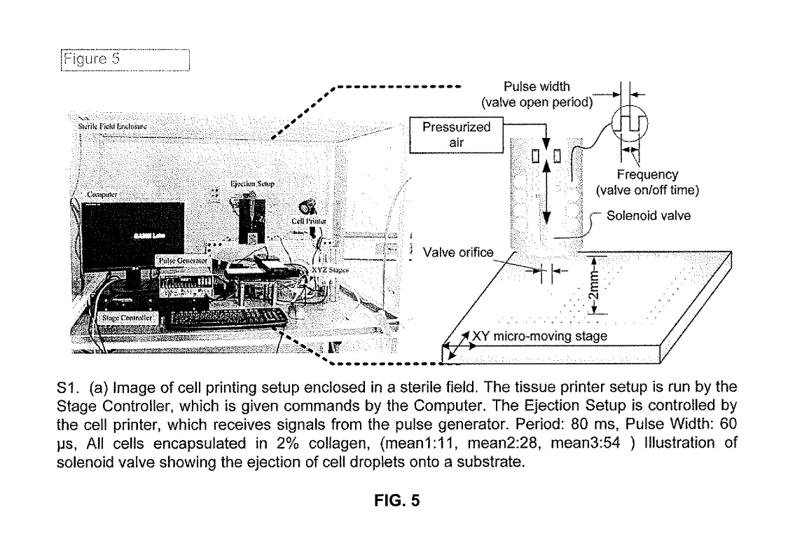

The bioengineering of a 3D bladder tissue is performed using a tissue printing setup as shown in FIG. 1. The ejection setup is controlled by the Cell Printer which receives signals from the pulse generator. Different initial concentrations were used to allow an understanding as to how reproducible cells per droplet are generated to create a tissue patch. The corresponding graph in FIG. 1b illustrates that as initial concentration increases, cells per droplet also increase. Variability in the printer path is illustrated by FIG. 2. The graph shows variation in the x and y coordinates for each droplet that contains smooth muscle cells. Smooth muscle cells encapsulated in collagen and printed in a unidirectional pattern were grown and stained with DAPI and actin. FIGS. 3a and 3b show that smooth muscle cells are viable and functional in a patch at day 2 and day 7 respectively. FIGS. 3e and 3f show SMC functional at day 14 in culture. In layering smooth muscle cells on top of another, cells encapsulated in 2% collagen were printed layer by layer. FIG. 4 (a) Cell distribution of 2D patch for 1, 5, 10 million cells/ml concentration. Each patch size is 3 mm by 10 mm, (b) μ±σ (average±STD) of each patches are 85±13, 114±42, 186±77, respectively. FIG. 5 illustrates smooth muscle layering and the corresponding focal images show the cells in their respective layers.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

The following references are incorporated herein by reference for any use: 1) F. Oberpenning, J. Meng, J. J. Yoo and A. Atala, Nat Biotechnol, 1999, 17, 149-155; 2) Y. Zhang, B. P. Kropp, H. K. Lin, R. Cowan and E. Y. Cheng, Tissue Engineering, 2004, 10, 181-187; 3) A. Atala, S. B. Bauer, S. Soker, J. J. Yoo and A. B. Retik, Lancet, 2006, 367, 1241-1246; 4) A. Atala, Curr Opin Pediatr, 2006, 18, 167-171; 5) A. L. Brown, T. T. Brook-Allred, J. E. Waddell, J. White, J. A. Werkmeister, J. A. Ramshaw, D. J. Bagli and K. A. Woodhouse, Biomaterials, 2005, 26, 529-543; 6) G. Christ, K. E. Andersson and A. Atala, Curr Urol Rep, 2007, 8, 95-99; 7) C. J. Koh and A. Atala, Minerva Ginecol, 2004, 56, 371-378; 8) S. Korossis, F. Bolland, E. Ingham, J. Fisher, J. Kearney and J. Southgate, Tissue Eng, 2006, 12, 635-644; 9) B. P. Kropp and J. B. Zwischenberger, Nat Clin Pract Urol, 2006, 3, 588-589; 10) S. Oottamasathien, K. Williams, O. E. Franco, J. C. Thomas, K. Saba, N. A. Bhowmick, A. Staack, R. T. Demarco, J. W. Brock, 3rd, S. W. Hayward and J. C. t. Pope, Dev Dyn, 2006, 235, 2795-2801; 11) M. A. Pattison, T. J. Webster and K. M. Haberstroh, J Biomater Sci Polym Ed, 2006, 17, 1317-1332; 12) A. Thapa, D. C. Miller, T. J. Webster and K. M. Haberstroh, Biomaterials, 2003, 24, 2915-2926; 13) S. Urakami, H. Shiina, H. Enokida, K. Kawamoto, N. Kikuno, T. Fandel, K. Vejdani, L. Nunes, M. Igawa, E. A. Tanagho and R. Dahiya, World J Urol, 2007, 25, 207-213; 14) Y. Zhang, D. Frimberger, E. Y. Cheng, H. K. Lin and B. P. Kropp, BJU Int, 2006, 98, 1100-1105; 15) Y. Zhang, B. P. Kropp, H. K. Lin, R. Cowan and E. Y. Cheng, Tissue Eng, 2004, 10, 181-187; 16) R. Langer and J. P. Vacanti, Science, 1993, 260, 920-926; 17) Stevens M. M., Qanadilo H. F., Langer R. and P. S. V., Biomaterials, 2004, 25, 887-894; 18) R. M. Nerem, Ann Biomed Eng, 1991, 19, 529-545; 19) L. E. Freed and G. Vunjak-Novakovic, Principles of Tissue Engineering, Landes, Austin, Tex., 1997; 20) V. I. S. G. N. Bancroft, J. V. D. Dolder, T. L. Sheffield, C. G. Ambrose, J. A. and A. G. M. Jansen, Proc. Natl. Acad. Sci, 2002, 99, 12600-12605; 21) K. F. L. S. Yang, Z. Du, C. K. Chua. The Design of Scaffolds for Use in Tissue and—. Engineering. Part II. Rapid Prototyping Techniques. Tissue Eng. 2002, Tissue Eng., 2002, 8, 1-11; 22) V. Mironov, T. Boland, T. Trusk, G. Forgacs and R. R. Markwald, Trends Biotechnol, 2003, 21, 157-161; 23) A. L. Valerie and N. B. Sangeeta, Biomed Microdevices, 2002, 4, 257; 24) D. G. Percin, Stanford University, 2002; 25) J. A. Barron, B. R. Ringeisen, B. J. S. H. Kim and D. B. Chrisey, Thin Solid Films, 2004, 453, 383; 26) T. Boland, V. Mironov, A. Gutowska, E. A. Roth and R. R. Markwald, Anat Rec A Discov Mol Cell Evol Biol, 2003, 272, 497-502; 27) R. S. Langer and J. P. Vacanti, Sci Am, 1999, 280, 86-89; 28) U. Demirci and M. Toner, Applied Physics Letters, 2006, 88; 29) M. S. Robert Chang, Jae Nam, M. S., and Wei Sun, Ph.D., Tissue Eng, 2008, 14, 157-166; 30) J. Nagatomi, F. DeMiguel, K. Torimoto, M. B. Chancellor, R. H. Getzenberg, and M. S. Sacks, Biochem Biophys Res Commun, 2005, 334, 1159-1164; 31) J. Nagatomi, K. K. Toosi, J. S. Grashow, M. B. Chancellor and M. S. Sacks, Annals Of Biomedical Engineering, 2005, 33, 1078-1089; 32) K. S. Heffernan, J. J. Sosnoff, C. A. Falls, K. K. Shinsako, S. Y. Jae, B. Fernhall, J Appl Physiol, 2008, 105, 109-113; 33) S. Havlin, S. V. Buldyrev, A. L. Goldberger, R. N. Mantegna, S. M. Ossadnik, C. K. Peng, M. Simons, H. E. Stanley, Chaos Solitons Fractals, 1995, 6, 171-201; 34) H. E. Stanley, S. V. Buldyrev, A. L. Goldberger, Z. D. Goldberger, S. Havlin, R. N. Mantegna, S. M. Ossadnik, C. K. Peng, M. Simons, Physica A, 1994, 205, 214-253; 35) H. E. Stanley, S. V. Buldyrev, A. L. Goldberger, J. M. Hausdorff, S. Havlin, J. Mietus, C -K Peng, F. Sciortino, M. Simons, Physica A, 1992, 191, 1-12; 36) C. K. Peng, S. V. Buldyrev, J. M. Hausdorff, S. Havlin, J. E. Mietus, M. Simons, H. E. Stanley, A. L. Goldberger, Integr Physiol Behav Sci, 1994, 29, 283-293; and 37) J. S. Perkiomaki, T. H. Makikallio, H. V. Huikuri, Clin Exp Hypertens, 2005, 27, 149-158.

1. A method for generating a three-dimensional tissue construct, the method comprising:
(a) providing a tissue scaffold;
(b) providing isolated smooth muscle cells;
(c) suspending the isolated cells in hydrogel;
(d) ejecting the cell/hydrogel suspension from a printer onto the tissue scaffold; and
(e) incubating the cells on the scaffold in media at 37° C.

2. The method of claim 1, wherein the scaffold is biologic or synthetic.

3. The method of claim 2, wherein the scaffold is made of carbon nanotube.

4. The method of claim 2, wherein the scaffold is made of collagen, fibrin, chitosan, or hyaluronic acid.

5. The method of claim 2, wherein the scaffold is made of linear aliphatic polyester.

6. The method of claim 2, wherein the scaffold is made of polylactic acid, polyglycolic acid, or polycaprolactone.

7. The method of claim 1, wherein the cells are suspended at a concentration of at least about 2.5×10⁵, at least about 5×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, or at least about 1.5×10⁷ cells/mL.

8. The method of claim 1, wherein the hydrogel is collagen.

9. The method of claim 1, wherein the hydrogel is extra cellular matrix.

10. The method of claim 1, wherein the concentration of hydrogel is at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL, at least about 80 mg/mL, at least about 90 mg/mL, or at least about 100 mg/mL.

11. The method of claim 1, wherein the tissue construct is a bladder.

12. The method of claim 1, wherein prior to step (d), cell-free hydrogel is placed onto the scaffold.

13. The method of claim 1, wherein step (d) is repeated one or more times.

14. A method of providing a tissue construct for implantation into a human, the method comprising;
(a) providing a tissue scaffold;
(b) providing isolated smooth muscle cells;
(c) suspending the isolated cells in hydrogel;
(d) ejecting the cell/hydrogel suspension from a printer onto the tissue scaffold;
(e) incubating the cells on the scaffold in media at 37° C.;
wherein the tissue construct is implanted into a human.
